(19) United States
(12) Patent Application Publication
(10) Pub. No.: US 2018/0104156 A1
(43) Pub. Date: Apr. 19, 2018

(54) METHOD AND SYSTEM OF PRODUCING A TEAT PRODUCT

(71) Applicant: Rosemary Mobbs, Coogee (AU)

(72) Inventor: Rosemary Mobbs, Coogee (AU)

(21) Appl. No.: 15/562,605

(22) PCT Filed: Apr. 1, 2016
(86) PCT No.: PCT/AU2016/050247

§ 371 Date: Sep. 28, 2017

(30) Foreign Application Priority Data
Apr. 1, 2015 (AU) 2015100425

(51) Int. Cl. A61J 11/00; B33Y 50/02; B33Y 80/00; B29C 64/393

(52) U.S. Cl. CPC A61J 11/005; B33Y 50/02; A61B 5/004; B29C 64/393; B33Y 80/00

(57) ABSTRACT

The present disclosure provides a method for producing a customised teat product for a subject, the method includes the initial step of performing a 3D scan of at least a nipple region of the subject's breast to create a 3D model of the nipple region. In the next step, a 3D product model for a teat product is created, incorporating a teat that corresponds in shape to at least the nipple region of the subject, based on the 3D model of the nipple region. In the final step, a physical teat product is generated based on the 3D product model using an additive or subtractive manufacturing apparatus.

### TECHNICAL FIELD

The present disclosure relates to a maternity teat product for use with baby bottles, soothers, nipple shields, and sippy cups and a method and system for producing the teat product. The maternity teat products produced may be used with human infants or the infants of other mammals, such as in zoos, animal sanctuaries and animal hospitals.

### BACKGROUND OF THE INVENTION

A number of products exist to assist new mothers with infants that involve the use of an imitation teat designed to imitate the nipple of the mother's breast. Such commercial teat products include baby bottle teats, soothers, nipple shields, and, for older infants, sippy cups.

Baby bottles are commonly used to feed infants and toddlers in lieu of breast feeding and comprise a bottle with a teat, typically made of silicone, latex, or rubber. Baby bottles are commonly used to feed infants a range of liquids, such as expressed breast milk, milk made from evaporated milk formula, artificial milk formula, or pediatric electrolyte solution. Infants may need to be bottle-fed for many different reasons, such as simple convenience, insufficient breast milk supply, nipple damage or mastitis.

Infant soothers (or pacifiers) are used to calm infants by providing an imitation teat for the infant to suck on, which simulates the act of breast feeding and can help calm an upset infant. The soother typically has a silicone, rubber or plastic teat attached to a mouth shield and handle.

Nipple shields are used to protect a feeding mother's nipples during breast feeding. They consist of a soft, thin nipple-shaped sheath that covers the areola and nipple, which helps protect against cracked and sore nipples. Use of a nipple shield can also assist with developing an infant's latching technique.

Use of commercial imitation teats in baby bottles, soothers, nipple shields and other imitation teat products, such as sippy cups, can result in an infant developing “nipple confusion”, in which an infant is reluctant to breastfeed and prefers to be bottle-fed milk. With increased use of baby bottles and other imitation teats, nipple confusion is becoming more and more prevalent and is having a significant impact on the rate of breastfeeding amongst new mothers.

Breastfeeding is widely acknowledged as providing substantial health benefits for infants and any decline in the rate of breastfeeding amongst new mothers may result in a consequent increase in the prevalence of certain health complications and allergies.

### OBJECT OF THE INVENTION

It is an object of the present invention to substantially overcome or at least ameliorate one or more of the above disadvantages, or to provide a useful alternative.

### SUMMARY OF THE INVENTION

In a first aspect, the present disclosure provides a method for producing a customised teat product for a subject, the method comprising:

performing a 3D scan of at least a nipple region of the subject's breast to create a 3D model of the nipple region;

creating a 3D product model for a teat product, incorporating a teat that corresponds in shape to at least the nipple region of the subject, based on the 3D model of the nipple region; and

generating a physical teat product based on the 3D product model using an additive or subtractive manufacturing apparatus.

In a preferred embodiment, the teat product is generated using a 3D printer. Preferably, the teat product is one of a baby bottle teat, a soother teat, a nipple shield, or a sippy cup teat. Further preferably, the nipple region includes at least the nipple and areola.

In a preferred embodiment, the teat product is formed from silicone. Preferably, the subject is a new mother or an expectant mother.

In a preferred embodiment, the 3D product model is altered to include an expandable section between a nipple portion and a main body portion. Preferably, the expandable section is in the form of a generally cylindrical and folded section that is expandable by pulling on the nipple portion to unfold the folded section.

In a further preferred embodiment, the 3D product model is altered to include a flange or lip around a periphery of the teat product.

In a second aspect, the present disclosure provides a system for producing a customised teat for a subject, the system comprising:

3D scanning apparatus adapted to scan at least a nipple region of the subject's breast, generate a 3D nipple model, and output image data representing the 3D nipple model;

a processor having a modelling module with software operable to allow a user to modify the 3D nipple model to create a 3D product model, the processor being adapted to output data representing a three-dimensional product design; and an additive or subtractive manufacturing apparatus adapted to generate a teat product based on the 3D product model.

In a third aspect, the present disclosure provides a teat product produced using the method or system of either the first or second aspect.

In a fourth aspect, the present disclosure provides a 3D modelling computing device comprising:

a memory for storing data and a computer program;

a processor coupled to said memory for executing said computer program stored in said memory; and

a modelling application forming part of said computing program, said modelling application including instructions for performing the method steps of:
receiving a 3D nipple model relating to a nipple region of a subject's breast;
modifying the 3D nipple model to create a 3D product model, based on user input, said 3D product model relating to a teat product.

In a preferred embodiment, the teat product is selected from the group consisting of: an infant soother teat, a bottle teat, a nipple shield, and a sippy cup teat.

Further preferably, one or more teat product templates are stored in the memory and the step of modifying the 3D nipple model to create a 3D product model comprises applying one of the teat product templates, selected by the user, to the 3D nipple model.

In a preferred embodiment, the step of modifying the 3D nipple model to create a 3D product model comprises altering the 3D nipple model to include an expandable section between a nipple portion and a main body portion.

Preferably, the expandable section is in the form of a generally cylindrical and folded section that is expandable by pulling on the nipple portion to unfold the folded section.

In a further preferred embodiment, the step of modifying the 3D nipple model to create a 3D product model comprises altering the 3D nipple model to include a flange or lip around a periphery of the teat product.

Preferably, the 3D product model is suitable as an input to an additive or subtractive manufacturing apparatus.

According to another aspect, the present disclosure provides an apparatus for implementing any one of the aforementioned methods.

According to another aspect, the present disclosure provides a computer program product including a computer readable medium having recorded thereon a computer program for implementing any one of the methods described above.

Other aspects of the present disclosure are also provided.

### BRIEF DESCRIPTION OF THE DRAWINGS

One or more embodiments of the present disclosure will now be described by way of specific example(s) with reference to the accompanying drawings, in which:

FIG. 1 is a flow diagram illustrating a method of producing a customised teat product;

FIG. 2 is a schematic representation of a system on which one or more embodiments of the present disclosure may be practised;

FIG. 3 is a schematic block diagram representation of a system that includes a general purpose computer on which one or more embodiments of the present disclosure may be practised; and

FIGS. 4a to 4c depict a customised teat product in different configurations.

### DETAILED DESCRIPTION OF THE PREFERRED EMBODIMENTS

The present disclosure provides a method and system for producing a customised maternity teat product. The term ‘teat product’, as it is used throughout this specification, refers to any product having an imitation nipple component, designed to be used in conjunction with the feeding of an infant. Examples of common teat products include baby bottle teats, soothers/pacifiers, nipple shields, and sippy cups.

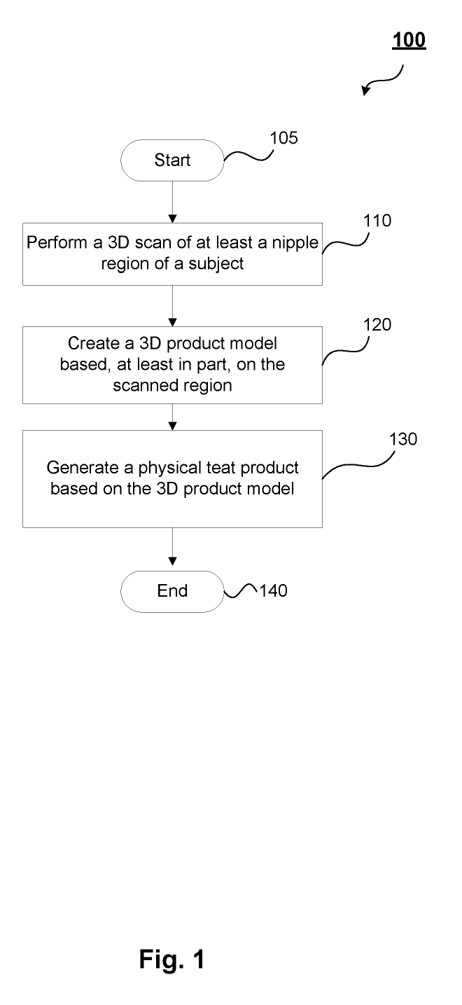

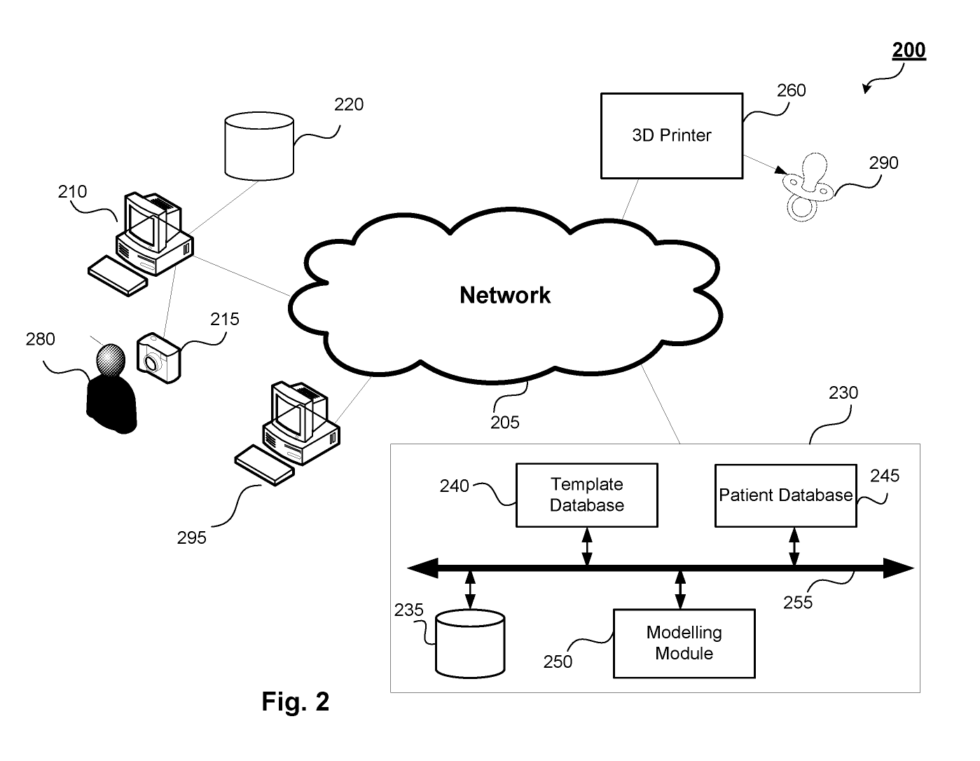

FIG. 1 is a flow diagram illustrating a method 100 and FIG. 2 depicts a system 200, both the method and system being for producing a customised teat product 290 for a given subject 280, usually a new or expectant mother. The subject 280 may be human or any other kind of mammal such as various primates.

As depicted in FIG. 1, the method 100 begins at a Start step 105 and proceeds to step 110, which performs a three-dimensional (3D) scan of at least a portion of a breast of the subject 280, including the nipple region of the breast. Typically, the nipple region scanned will include both the nipple and areola.

As depicted in FIG. 2, a 3D scanning device 210, having a camera 215 and a memory 220, is used to collect 3D data on the shape of the nipple region of the breast of the subject 280. The 3D scanner 210 uses the 3D data to create a 3D model of the scanned nipple region, which is stored in the memory 220 as a 3D nipple model. It will be appreciated that the camera 215 and memory 220 may be integral to the scanning device 210 or external peripherals connected to the scanning device 210.

Returning to FIG. 1, control passes from step 110 to step 120, which uses the 3D model of the scanned nipple region to create a 3D product model of a teat product by altering the 3D model of the scanned nipple region. For example, in one embodiment, the 3D model is modified to create a baby bottle teat by truncating the 3D model, as appropriate, and adding a circumferential flange.

As depicted in FIG. 2, the 3D model of the scanned nipple region, or output image data representative of the 3D nipple model, is transmitted from the memory 220 of the scanning device 210 via a network 205 to a modelling computer 230 having a memory 235, a template database 240, a patient database 245, and a modelling module 250, all of which communicate via a bus 255. The 3D model of the scanned nipple region is stored in the patient database 245 and, by way of the modelling module 250, the modelling computer 230 allows a user to create a 3D product model using the 3D model of the scanned nipple region.

A user accesses the modelling computer 230, either directly or by using the scanning device 210 or a computing device 295 coupled to the network 205, in order to provide user input to the modelling module 250. In one embodiment, the user uses an input device of the scanning device 210 or the computing device 295 to transmit user input to the modelling module 250 of the modelling computer 230. The modelling module 250 receives the user input to create the 3D product model.

The template database 240 optionally provides one or more teat product 290 templates, such as a template for the flange of a baby bottle teat, which facilitate the creation of the 3D product model. In an alternative implementation, the scanning device 210 and modelling computer 230 are integrated into a single computing device.

Returning to FIG. 1, control passes from step 120 to step 130, which generates a physical teat product 290 based on the 3D product model. For example, in one embodiment, a physical teat product 290 is produced from an additive manufacturing device, such as a 3D printer, based on the 3D product model and, for example, using silicone as the printing medium. In such an embodiment, the 3D printer lays down successive layers of silicone in accordance with the 3D product model to produce the physical teat product 290. Other additive or subtractive manufacturing techniques, such as continuous liquid interface production (CLIP) may also be employed to produce the physical teat product 290 from the 3D product model.

As depicted in FIG. 2, the 3D product model is transmitted from the modelling computer 230 via the network 205 to an additive manufacturing device 260, which uses the 3D product model to produce the teat product 290. In this example, the teat product 290 is an infant soother or pacifier. However, it will be appreciated that the teat product 290 may be embodied as any teat product. Control passes from step 130 to an End step 140 and the method 100 terminates.

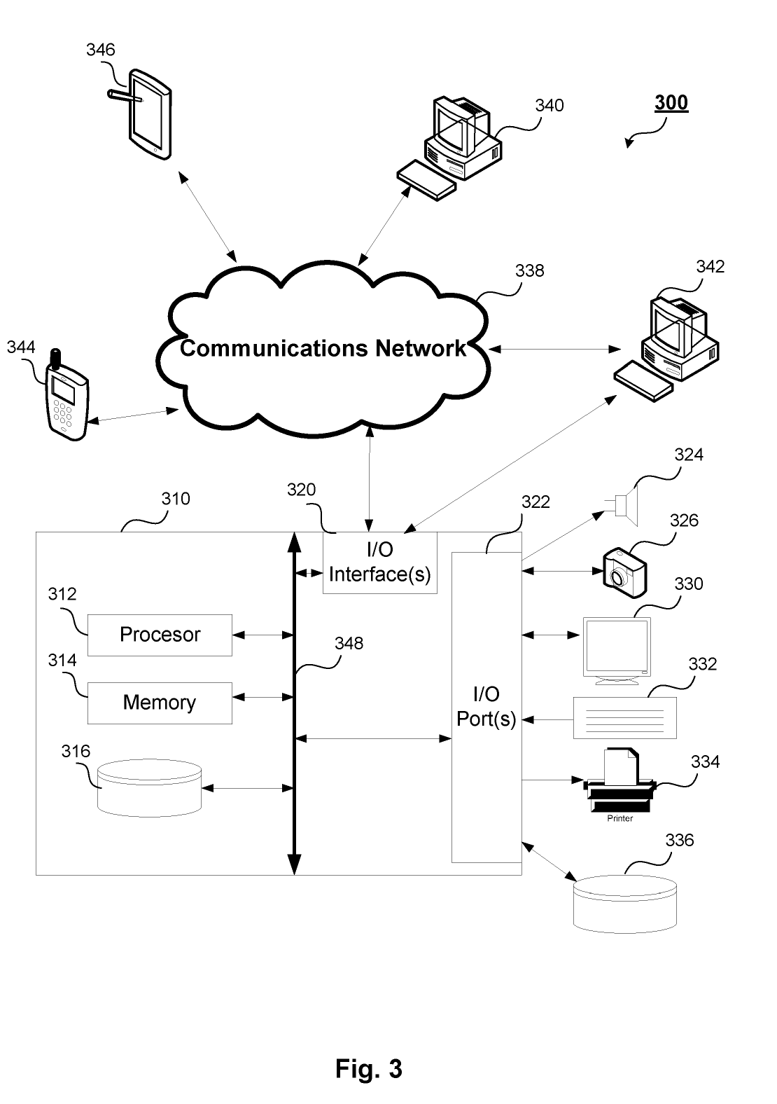

The system for producing a teat product 290 of the present disclosure may be practised using one or more computing devices, such as a general purpose computer or computer server. FIG. 3 is a schematic block diagram of a system 300 that includes a general purpose computer 310. The general purpose computer 310 includes a plurality of components, including: a processor 312, a memory 314, a storage medium 316, input/output (I/O) interfaces 320, and input/output (I/O) ports 322. Components of the general purpose computer 310 generally communicate using one or more buses 348.

The memory 314 may be implemented using Random Access Memory (RAM), Read Only Memory (ROM), or a combination thereof. The storage medium 316 may be implemented as one or more of a hard disk drive, a solid state “flash” drive, an optical disk drive, or other storage means. The storage medium 316 may be utilised to store one or more computer programs, including an operating system, software applications, and data. In one mode of operation, instructions from one or more computer programs stored in the storage medium 316 are loaded into the memory 314 via the bus 348. Instructions loaded into the memory 314 are then made available via the bus 348 or other means for execution by the processor 312 to implement a mode of operation in accordance with the executed instructions.

One or more peripheral devices may be coupled to the general purpose computer 310 via the I/O ports 322. In the example of FIG. 3, the general purpose computer 310 is coupled to each of a speaker 324, a camera 326, a display device 330, an input device 332, a printer 334, and an external storage medium 336. The speaker 324 may be implemented using one or more speakers, such as in a stereo or surround sound system. In the example in which the general purpose computer 310 is utilised to implement the scanning device 210, one or more peripheral devices may relate to the camera 215 and memory 220 connected to the I/O ports 322. In the example in which the general purpose computer 310 is utilised to implement the modelling computer 230, scanning device 210, or computing device 295, one or more peripheral devices may relate to a keyboard, touch tablet, mouse, or other input device for providing user input to the modelling module 250.

The camera 326 may be a webcam, or other still or video digital camera, and may download and upload information to and from the general purpose computer 310 via the I/O ports 322, dependent upon the particular implementation. For example, images recorded by the camera 326 may be uploaded to the storage medium 316 of the general purpose computer 310. Similarly, images stored on the storage medium 316 may be downloaded to a memory or storage medium of the camera 326. The camera 326 may include a lens system, a sensor unit, and a recording medium.

The display device 330 may be a computer monitor, such as a cathode ray tube screen, plasma screen, or liquid crystal display (LCD) screen. The display 330 may receive information from the computer 310 in a conventional manner, wherein the information is presented on the display device 330 for viewing by a user. The display device 330 may optionally be implemented using a touch screen to enable a user to provide input to the general purpose computer 310. The touch screen may be, for example, a capacitive touch screen, a resistive touchscreen, a surface acoustic wave touchscreen, or the like.

The input device 332 may be a keyboard, a mouse, a stylus, drawing tablet, or any combination thereof, for receiving input from a user. The external storage medium 336 may include an external hard disk drive (HDD), an optical drive, a floppy disk drive, a flash drive, or any combination thereof and may be implemented as a single instance or multiple instances of any one or more of those devices. For example, the external storage medium 336 may be implemented as an array of hard disk drives.

The I/O interfaces 320 facilitate the exchange of information between the general purpose computing device 310 and other computing devices. The I/O interfaces may be implemented using an internal or external modem, an Ethernet connection, or the like, to enable coupling to a transmission medium. In the example of FIG. 3, the I/O interfaces 322 are coupled to a communications network 338 and directly to a computing device 342. The computing device 342 is shown as a personal computer, but may be equally practised using a smartphone, laptop, or a tablet device. Direct communication between the general purpose computer 310 and the computing device 342 may be implemented using a wireless or wired transmission link.

The communications network 338 may represent the network 205 of FIG. 2 and may be implemented using one or more wired or wireless transmission links and may include, for example, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a mobile telephone cellular network, a short message service (SMS) network, or any combination thereof. The general purpose computer 310 is able to communicate via the communications network 338 to other computing devices connected to the communications network 338, such as the mobile telephone handset 344, the touchscreen smartphone 346, the personal computer 340, and the computing device 342.

One or more instances of the general purpose computer 310 may be utilised to implement the scanning device 210 and modelling computer 230 to implement a 3D modelling system for producing a customised teat product in accordance with the present disclosure. In such an embodiment, the memory 314 and storage 316 are utilised to store data relating to 3D modelling software, teat product templates, and patient data. Software for implementing the 3D modelling system is stored in one or both of the memory 314 and storage 316 for execution on the processor 312. The software includes computer program code for implementing method steps in accordance with the method of FIG. 1 described herein.

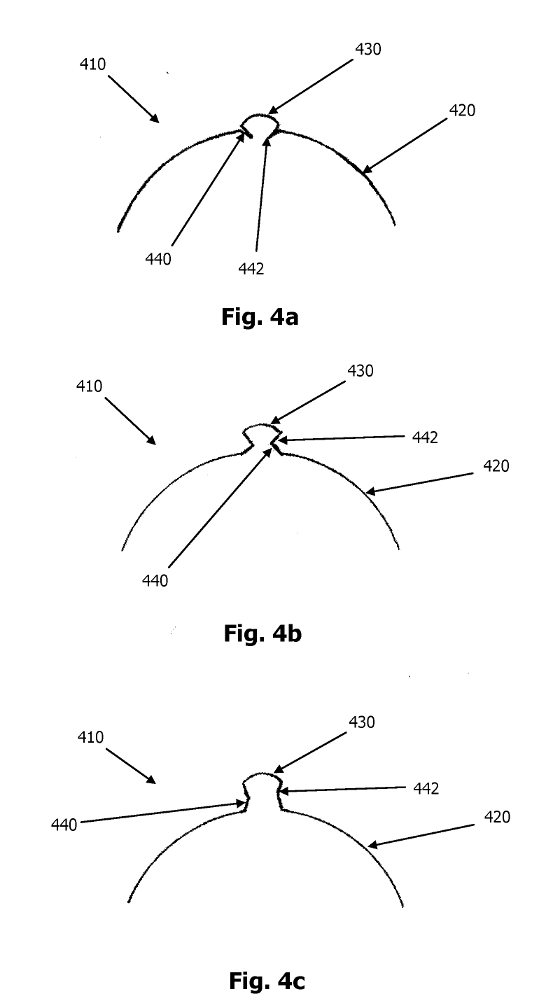

An embodiment of a teat product formed by the above process is depicted in FIGS. 4a to 4c. The teat product 410 depicted in FIG. 4a has a main body 420 and a nipple portion 430 that has been 3D-printed based on a 3D nipple model created from a scan of a subject's nipple region. In this embodiment, the 3D nipple model has been altered to include an expandable section 440 between the nipple portion 430 and the main body 420. The main body 420 typically includes an areola region adjacent to the expandable section 440. The expandable section 440 includes a generally cylindrical section with a central fold line 442 that is adapted to expand and collapse in a generally concertina-type fashion between the collapsed configuration depicted in FIG. 4a and the expanded configuration shown in FIG. 4c. FIG. 4b depicts a partially collapsed/expanded configuration. In this embodiment, the teat product is produced by 3D printing in the collapsed configuration depicted in FIG. 4a and is therefore biased to the collapsed configuration. When an infant sucks on the teat product, the expandable section 440 expands into the expanded configuration shown in FIG. 4c and then collapses back to the collapsed configuration shown in FIG. 4a when the infant stops sucking.

### INDUSTRIAL APPLICABILITY

The arrangements described are applicable to the medical and health care industries and particularly for the postnatal health care and infant (human and other mammalian) product industries.

The foregoing describes only some embodiments of the present invention, and modifications and/or changes can be made thereto without departing from the scope and spirit of the invention, the embodiments being illustrative and not restrictive.

In the context of this specification, the word “comprising” and its associated grammatical constructions mean “including principally but not necessarily solely” or “having” or “including”, and not “consisting only of”. Variations of the word “comprising”, such as “comprise” and “comprises” have correspondingly varied meanings.

As used throughout this specification, unless otherwise specified, the use of ordinal adjectives “first”, “second”, “third”, “fourth”, etc., to describe common or related objects, indicates that reference is being made to different instances of those common or related objects, and is not intended to imply that the objects so described must be provided or positioned in a given order or sequence, either temporally, spatially, in ranking, or in any other manner.

Although the invention has been described with reference to specific examples, it will be appreciated by those skilled in the art that the invention may be embodied in many other forms.

1. A method for producing a customised teat product for a subject, the method comprising:
performing a 3D scan of at least a nipple region of the subject's breast to create a 3D model of the nipple region;
creating a 3D product model for a teat product, incorporating a teat that corresponds in shape to at least the nipple region of the subject, based on the 3D model of the nipple region; and
generating a physical teat product based on the 3D product model using an additive or subtractive manufacturing apparatus.

2. The method of claim 1 wherein the teat product is generated using a 3D printer.

3. The method of claim 1 wherein the teat product is one of a baby bottle teat, a soother teat, a nipple shield, and a sippy cup teat.

4. The method of claim 1 wherein the nipple region includes at least the nipple and areola.

5. The method of claim 1 wherein the teat product is formed from silicone.

6. The method of claim 1 wherein the subject is a new mother or an expectant mother.

7. The method of claim 1 wherein the 3D product model is altered to include an expandable section between a nipple portion and a main body portion.

8. The method of claim 7 wherein the expandable section is in the form of a generally cylindrical and folded section that is expandable by pulling on the nipple portion to unfold the folded section.

9. The method of claim 1 wherein the 3D product model is altered to include a flange or lip around a periphery of the teat product.

10. A system for producing a customised teat for a subject, the system comprising:
3D scanning apparatus adapted to scan at least a nipple region of the subject's breast, generate a 3D nipple model, and output image data representing the 3D nipple model;
a modelling computer having a modelling module with software operable to allow a user to modify the 3D nipple model to create a 3D product model, the processor being adapted to output data representing a three-dimensional product design; and
an additive or subtractive manufacturing apparatus adapted to generate a teat product based on the 3D product model.

11. The system of claim 10, wherein the manufacturing apparatus is a 3D printer.

12. A teat product produced using the method of claim 1.

13. A 3D modelling computing device comprising:
a memory for storing data and a computer program;
a processor coupled to said memory for executing said computer program stored in said memory; and
a modelling application forming part of said computing program, said modelling application including instructions for performing the method steps of:
receiving a 3D nipple model relating to a nipple region of a subject's breast;
modifying the 3D nipple model to create a 3D product model, based on user input, said 3D product model relating to a teat product.

14. The 3D modelling computing device of claim 13 wherein the teat product is selected from the group consisting of: an infant soother teat, a bottle teat, a nipple shield, and a sippy cup teat.

15. The 3D modelling computing device of claim 13 wherein one or more teat product templates are stored in the memory and the step of modifying the 3D nipple model to create a 3D product model comprises applying one of the teat product templates, selected by the user, to the 3D nipple model.

16. The 3D modelling computing device of claim 13 wherein the step of modifying the 3D nipple model to create a 3D product model comprises altering the 3D nipple model to include an expandable section between a nipple portion and a main body portion.

17. The 3D modelling computing device of claim 16 wherein the expandable section is in the form of a generally cylindrical and folded section that is expandable by pulling on the nipple portion to unfold the folded section.

18. The 3D modelling computing device of claim 13 wherein the step of modifying the 3D nipple model to create a 3D product model comprises altering the 3D nipple model to include a flange or lip around a periphery of the teat product.

19. The 3D modelling computing device of claim 13 wherein the 3D product model is suitable as an input to an additive or subtractive manufacturing apparatus.

20. A teat product produced using the system of claim 10.
